(19) United States
(12) Patent Application Publication
(10) Pub. No.: US 2002/0123480 A1
(43) Pub. Date: Sep. 5, 2002

(54) Methods and compositions utilizing 2'-5' oligoadenylates in the treatment of disorders of cell proliferation

(76) Inventors: Clark M. Edson, Watertown, MA (US); Samuel K. Ackerman, Weston, MA (US)

Correspondence Address: Ivor R. Elrifi, MINTZ, LEVIN, COHN, FERRIS,, GLOVSKY and POPEO, P.C., One Financial Center, Boston, MA, 02111, US

(21) Appl. No.: 10/000,498

(22) Filed: Oct. 23, 2001

Related U.S. Application Data
(60) Provisional application No. 60/242,802, filed on Oct. 24, 2000.

(51) Int. Cl. A61K 48/00

(52) U.S. Cl. 514/44

(57) ABSTRACT

2′,5′-oligoadenylate-2′,3′-cyclophosphates having the chemical structure of Formula I:

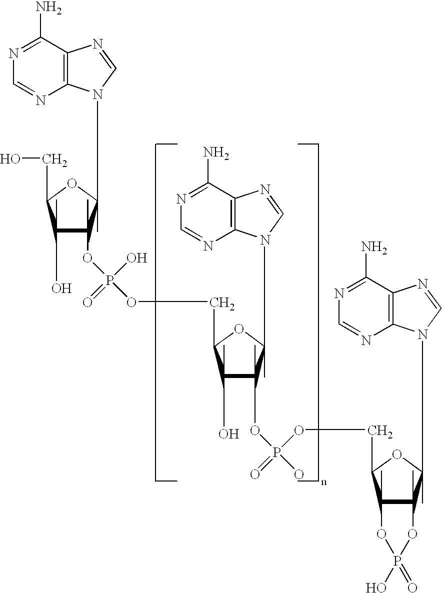

wherein 0≦n≦10, or the pharmaceutically acceptable salts thereof, inhibit cell proliferation and tumor cell growth, and are therefore useful in methods and compositions for the treatment of disorders of cell proliferation, including cancer, and also in the reduction of tumor size.

### RELATED APPLICATION

The present application claims priority under 35 U.S.C. §120 to U.S. provisional patent application serial no. 60/242,802, which was filed on Oct. 24, 2000 and which is incorporated by reference herein in its entirely.

### FIELD OF THE INVENTION

This invention relates to methods and compositions for treating cancer and other disorders of cell proliferation.

### BACKGROUND OF THE INVENTION

In 1998, cancer was the second leading cause of death in the United States behind heart disease. Unfortunately, the number of deaths from cancer is projected to increase with time. See D. Longo, in Harrison's Principles of Internal Medicine 14th Edition, pp. 492-3 (McGraw-Hill, New York, 1998). Surgery and radiotherapy may be curative if the disease is detected early, but current drug therapies for metastatic disease are mostly palliative and seldom offer a long-term cure. Even with the new chemotherapies entering the market, improvement in patient survival is measured only in months rather than in years. The use of presently known cancer treatments (e.g., surgery, radiation therapy, chemotherapy, etc.) results in the cure or remission of over 50% of patients diagnosed with cancer. However, the need for additional forms of treatment is ever-present, and the development of new antineoplastic agents, as well as therapies in the treatment of resistant tumors, remains a primary goal of medical research.

While cancer is likely the most devastating of the disorders of cell proliferation, other non-cancer disorders of cell proliferation such as myelodysplastic disorders, cervical and other hyperplasias, Gardner syndrome, hyperproliferative arterial stenosis and viral-induced hyperproliferative diseases may also pose medically grave threats to health and life. Accordingly, there is need for compounds and methods of treating these disorders of cell proliferation.

Numerous 2′,5′-oligoadenylate compounds (also referred to in the art as 2-5A compounds) and their analogs are known. See e.g., U.S. Pat. Nos. 5,955,446 and 5,739,013 to Budowsky, et at.).; U.S. Pat. Nos. 4,990,498, 5,188,897, 4,464,359, 4,859,768, and 5,550,111 to Suhadolnik et al.).; U.S. Pat. No. 5,571,799 to Tkachuk et al., U.S. Pat. No. 4,515,781 to Torrence et al.; U.S. Pat. No. 4,476,301 to Imbach et al.; and U.S. Pat. No. 4,981,957 to Lebteu et al. However, the successful use in human cells of 2′,5′-oligoadenylate compounds (e.g., 2′,5′-otigoadenytate-2′,3′-cyclophosphates) in the treatment of disorders of cell proliferation has heretofore not been illustrated.

### SUMMARY OF THE INVENTION

The present inventors have discovered that 2′,5′-oligoadenylate-2′,3′-cyclophosphates having the chemical structure of Formula I inhibit cell proliferation and tumor cell growth. Formula I is as follows:

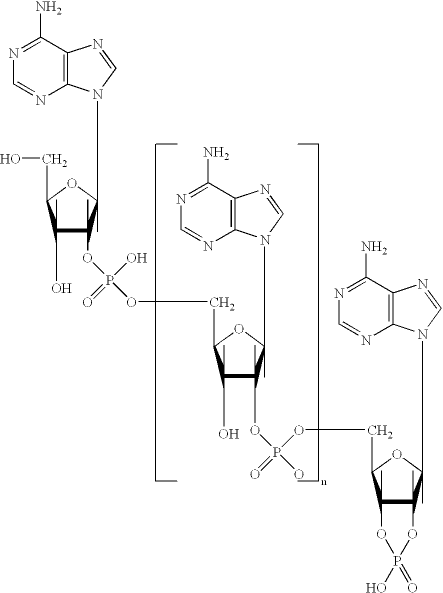

wherein n is an integer from zero to ten, inclusive (i.e., 0≦n≦10).

Accordingly, a first aspect of the present invention is a method of treating a disorder of cell proliferation (e.g., cancer) in a subject in need of such treatment, by administering to the subject a compound of Formula I or a pharmaceutically acceptable salt thereof, in an amount sufficient to treat the disorder.

A second aspect of the invention is a method of treating cancer in a subject in need of such treatment, by administering to the subject a compound of Formula I or a pharmaceutically acceptable salt thereof, in an amount sufficient to treat the cancer.

A third aspect of the invention is a is a method of reducing tumor size in a subject in need of such treatment, by administering to the subject a compound of Formula I or a pharmaceutically acceptable salt thereof, in an amount sufficient to reduce the size of the tumor.

A fourth aspect of the present invention is the use of a compound of Formula I for the preparation of a medicament for the treatment of cancer and other diseases of cell proliferation.

The foregoing and other aspects of the present invention are explained in detail in the specification set forth below.

### DETAILED DESCRIPTION OF THE PREFERRED EMBODIMENTS

The present invention will now be described more fully hereinafter, wherein preferred embodiments of the invention are disclosed. This invention may, however, be embodied in different forms and should not be construed as limited to the embodiments set forth herein. Rather, these embodiments are provided so that this disclosure will be thorough and complete, and will fully convey the scope of the invention to those skilled in the art.

The terminology used in the description of the invention herein is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise. Unless otherwise defined, all technical and scientific terms used herein have the same meaning as commonly understood by one of ordinary skill in the art to which this invention belongs. All publications, patent applications, patents, and other references mentioned herein are incorporated by reference in their entirety.

The present invention is suitable for both medical and veterinary uses. Suitable subjects include, but are not limited to, mammalian and avian subjects; preferably, mammalian subjects; more preferably human, monkeys, pigs, cattle, dogs, horses, cats, sheep, and goats; and most preferably human subjects. The methods and compounds of the present invention relate to the anti-proliferative activity of compounds of Formula I:

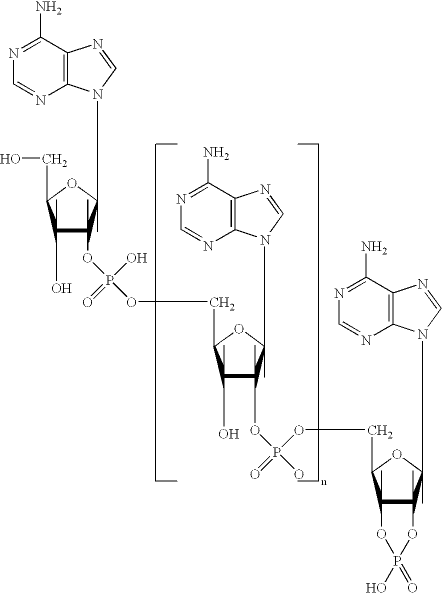

wherein n is an integer from zero to ten, inclusive (i.e., 0≦n≦10). In a preferred embodiment, n is greater than zero. In a more preferred embodiment, n is one or two. Compounds of Formula I and the pharmaceutically acceptable salts thereof are referred to herein as “active compounds.” Preferred active compounds of the present invention include, but are not limited to, 2′,3′-cyclophosphateadenylyl(2′,5′)adenylyl-(2′,5′)adenosine; 2′,3′-cyclophosphateadenylyl(2′,5′)adenylyl(2′,5′)adenylyl-(2′,5′)adenosine; and 2′,3′-cyclophosphateadenylyl(2′,5′)adenosine. Compounds of Formula I and methods of synthesizing the same are set forth in U.S. Pat. No. 5,739,013 to Budowsky et al., the disclosure of which is incorporated herein by reference in its entirety.

In methods of the present invention, the active compounds of Formula (I) may be administered by themselves or in the form of their pharmaceutically acceptable salts, e.g., an alkali metal salt such as sodium or potassium, an alkaline earth metal salt, or an ammonium and tetraalkyl ammonium salt, NX₄⁺ (wherein X is generally a C1-C4 alkyl group). Pharmaceutically acceptable salts are salts that retain the desired biological activity of the compound of Formula I but do which not impart undesired toxicological effects.

The active compounds are useful in the treatment and prevention of disorders of cell proliferation, including cancer. The treatments employed herein may be used to inhibit growth and/or to induce cytotoxicity in target cells, which are generally hyperproliferative cells (including tumors, cancers, and neoplastic tissue, along with pre-malignant and non-neoplastic or non-malignant hyperproliferative cells).

As used herein, the term “cancer” has its understood meaning in the art, for example, an uncontrolled growth of tissue that has the potential to spread to distant sites of the body (i.e., metastasize). See R. Fenton and D. Longo, in Harrison's Principles of Internal Medicine 15th Edition, p. 505 (McGraw-Hill, New York, 1998). The term “cancer” is intended to encompass both tumor-forming or non-tumor forming cancers. The methods and compositions of the present invention may be used to treat both a primary cancer and to prevent metastasis. As used herein, the term “cancer cell” is also intended to encompass those cells referred to as “pre-cancerous,” i.e., cells that contain mutated or damaged DNA or other components, which mutations or damage are likely to cause the cell to develop into a cancer cell.

Exemplary cancers include, but are not limited to, brain cancer, colon cancer, renal cancer, liver cancer, breast cancer, lung cancer, prostate cancer, ovarian cancer, melanoma, skin cancer (e.g., carcinoma, melanoma) and the like. As used herein, the term “tumor” is intended to mean an abnormal mass of cells within a multicellular organism. Generally, the growth of the abnormal cells of the tumor exceeds and is uncoordinated with that of normal cells. Furthermore, the abnormal growth of tumor cells generally persists in an abnormal (i.e., excessive) manner after the cessation of stimuli that originally caused the abnormality in the growth of the cells. Tumors can be malignant or benign. In a preferred embodiment, a cancer treated by the method of the present invention is skin cancer, preferably a skin carcinoma. In a more preferred embodiment, a cancer treated by the method of the present invention is a skin carcinoma selected from the group consisting of basal cell carcinoma and squamous cell carcinoma.

By the terms “treating cancer” or “treatment of cancer,” it is intended that the severity of the cancer is reduced or the cancer is partially or entirely eliminated, or that tumor size is reduced or that the tumor is partially or entirely eliminated, as compared to that which would occur in the absence of treatment. Alternatively, these terms are intended to mean that metastasis of the cancer is reduced or eliminated, as compared to that which would occur in the absence of treatment. The term “treating cancer” may also mean that the cell proliferation is decreased, as compared to that which would occur in the absence of treatment. Accordingly, “treatment” of a disorder of cell proliferation, particularly hyperproliferative disorders, refers to methods of killing, inhibiting or slowing the growth or increase in size of a body or population of abnormally proliferative cells or tumor or cancerous growth, reducing the number of cells in the population of abnormally proliferative cells, or preventing the spread of abnormally proliferative cells to other anatomic sites, as well as reducing the size of a growth of abnormally proliferative cells. As used herein, “treatment” is not necessarily meant to imply a cure or a complete abolition of a cancer, an abnormally proliferative growth or any other manifestation of a disorder of cell proliferation. As used herein, a treatment effective amount is an amount effective to result in the killing, the slowing of the rate of growth of abnormally proliferative cells, the decrease in size of a body of abnormally proliferative cells, and/or the reduction in number of abnormally proliferative cells.

By the terms “prevention of cancer” or “preventing cancer” it is intended that the inventive methods eliminate or reduce the incidence or onset of cancer, as compared to that which would occur in the absence of treatment. Alternatively stated, the present methods slow, delay, control, or decrease the likelihood or probability of cancer in the subject, as compared to that which would occur in the absence of treatment. In one such embodiment of the present invention, the methods and/or nutritional compositions of the present invention are administered to a subject with a likelihood of having pre-cancerous mutations in certain cells (e.g., a heavy smoker with a high likelihood of having mutations in lung cells) in order to prevent or delay the onset of lung cancer.

The methods and compositions of the present invention can be used in subjects who have already been diagnosed with cancer. As an alternative embodiment, the present invention can be carried out with individuals at risk for developing cancer. At-risk individuals include, but are not limited to, individuals with a family history of cancer, individuals who have previously been treated for cancer, individuals who have been exposed to carcinogens (e.g., heavy smokers), individuals exposed to medications or medical treatments associated with the development of cancer (e.g., estrogens or radiation therapy), individuals determined to have an increased likelihood of developing cancer by genetic testing, and individuals presenting any other clinical indicia suggesting that they have an increased likelihood of developing cancer. Alternatively stated, an at-risk individual is any individual who is believed to be at a higher risk than the general population for developing cancer.

The methods and compositions of the present invention are also useful in the treatment and prevention of non-cancer disorders of cell proliferation. These diseases include, but are not limited to, benign tumors, hyperplasias, hyperpigmentation of the skin, psoriasis, and any other disorder wherein cell proliferation is uncontrolled, and control of such proliferation is desired. Additional examples of premalignant or non-neoplastic or non-malignant disorders of cell proliferation (generally, hyperproliferative disorders) include, but are not limited to, myelodysplastic disorders, cervical carcinoma-in-situ, familial intestinal polyposes such as Gardner syndrome, oral leukoplakias, histiocytoses, keloids, hemangiomas, hyperproliferative arterial stenosis, inflammatory arthritis, hyperkeratoses and papulosquamous eruptions including arthritis. Also included in the scope of disorders that may be treated by the methods of the present invention are virus-induced hyperproliferative diseases such as human papilloma virus-induced disease (e.g., lesions caused by human papilloma virus infection), Epstein-Barr virus-induced disease, scar formation, genital warts, cutaneous warts, and the like. In a preferred embodiment, the disorder of cell proliferation treated by the methods and compositions of the present invention is human papilloma virus-induced disease.

Active compounds of the present invention may be administered in combination with other compounds that are already known in the art to be useful in the treatment of a disorder of cell proliferation, such as cancer. Numerous compounds useful in the treatment of cancer are set forth in U.S. Pat. No. 5,919,816 to Hausheer et al., the disclosure of which is incorporated herein by reference. As used herein, the administration of two or more compounds “in combination” means that the two compounds are administered closely enough in time that the presence of one alters the biological effects of the other. The two compounds may be administered simultaneously or sequentially. Simultaneous administration may be carried out by mixing the compounds prior to administration, or by administering the compounds at the same point in time but at different anatomic sites or using different routes of administration. The phrases “concurrent administration,” “administration in combination,” “simultaneous administration” or “administered simultaneously” as used herein, interchangeably connote that the compounds are administered at the same point in time or immediately following one another. In the latter case, the two compounds are administered at times sufficiently close that the results observed are indistinguishable from those achieved when the compounds are administered at the same point in time.

The active compounds and salts described for the treatment of the disorders described herein can be administered via any medically or veterinary accepted mode of administration, including parenteral (including subcutaneous, intradermal, intramuscular, intravenous and intraarticular), inhalation, oral, rectal, topical (including buccal, sublingual, dermal, vaginal, intraocular and administration by iontophoresis), and transdermal administration. In a preferred embodiment of the invention, the active compounds are administered topically or transdermally.

Any pharmaceutically acceptable mode of administration can be used, including solid, semi-solid or liquid dosage forms, such as, for example, tablets, suppositories, pills, capsules, powders, liquids, suspensions, or the like, preferably in unit dosage forms suitable for single administration of precise dosages, or in sustained or controlled release dosage forms for the prolonged administration of the compound at a predetermined rate. The compositions will typically include a conventional pharmaceutical carrier or excipient and an active compound or the pharmaceutically acceptable salts thereof and, in addition, may include other medicinal agents, pharmaceutical agents, carriers, adjuvants, etc.

For solid compositions, conventional non-toxic solid carriers include, for example, pharmaceutical grades of mannitol, lactose, cellulose, cellulose derivatives, sodium crosscarmellose, starch, magnesium stearate, sodium saccharin, talcum, glucose, sucrose, magnesium carbonate, and the like may be used. The active compound as defined above may be formulated as suppositories using, for example, polyalkylene glycols, acetylated triglycerides and the like, as the carrier. Liquid pharmaceutically administrable compositions can, for example, be prepared by dissolving, dispersing, etc. an active compound as defined above and optional pharmaceutical adjuvants in a carrier, such as, for example, water, saline, aqueous dextrose, glycerol, ethanol, and the like, to thereby form a solution or suspension. Pharmaceutical compositions useful in embodiments of the present invention may be lyophilized using techniques well known in the art.

If desired, the pharmaceutical composition to be administered may also contain nontoxic auxiliary substances such as wetting or emulsifying agents and the like, for example, sodium acetate, sorbitan monolaurate, triethanolamine sodium acetate, sorbitan monolaurate, triethanolamine oleate, etc. Actual methods of preparing such dosage forms are known, or will be apparent, to those skilled in this art; for example, see Remington's Pharmaceutical Sciences, Nineteenth Edition (Mack Publishing Company, Easton, Pennsylvania, USA 1995). The composition or formulation to be administered will, in any event, contain a quantity of the active compound(s) in an amount effective to treat the disorder.

Compositions of the present invention may also comprise other non-toxic additives such as preservatives, antioxidants, pH buffering agents, surface active agents, and the like. In particular, useful pH-buffering agents include acids, such as hydrochloric acid, bases or buffers, such as sodium lactate, sodium acetate, sodium phosphate, sodium citrate, sodium borate, or sodium gluconate. Further, the compositions may contain anti-microbial preservatives. Useful anti-microbial preservatives include methylparaben, propylparaben, and benzyl alcohol. The anti-microbial preservative is typically employed when the formulation is placed in a dosage form (e.g., a vial) designed for multidose use.

The active compounds of the present invention are preferably formulated for administration as topical compositions adapted for use in mammals. These compositions may be presented for use in any conventional manner with the aid of any of a wide variety of pharmaceutical carriers or vehicles. For such topical administration, a pharmaceutically acceptable non-toxic formulation can take the form of a semisolid, liquid, or solid, such as, for example, gels, ointments, pastes, creams, lotions, solutions, suspensions, ointments, powders, sprays, inhalants, patches (e.g., for transdermal administration) or the like. The topical delivery of the active compounds of the present invention is an aspect of the present invention. This treatment regimen is suitable either for the systemic administration of the pharmaceutical agent or for localized therapy, i.e., directly to pathological or diseased tissue.

Topical preparations can be prepared by combining an active compound of the present invention with conventional pharmaceutical diluents and carriers commonly used in topical dry, liquid, cream and aerosol formulations. Ointment and creams may, for example, be formulated with an aqueous or oily base with the addition of suitable thickening and/or gelling agents. Such bases may include water and/or an oil such as liquid paraffin or a vegetable oil such as peanut oil or castor oil. Carriers and diluents include but are not limited to ethanol, glycerol, water, and the like as known to those skilled in the art. Thickening agents which may be used according to the nature of the base include soft paraffin, aluminum stearate, propylene carbonate, diisopropyl adipate, cetostearyl alcohol, propylene glycol, polyethylene glycols, woolfat, hydrogenated lanolin, beeswax, and the like.

Lotions may be formulated with an aqueous or oily base and will, in general, also include one or more of the following: stabilizing agents, emulsifying agents, dispersing agents, suspending agents, thickening agents, coloring agents, perfumes, and the like.

Powders may be formed with the aid of any suitable powder base, e.g., talc, lactose, starch, and the like. Drops may be formulated with an aqueous base or non-aqueous base also comprising one or more dispersing agents, suspending agents, solubilizing agents, and the like.

The ointments, pastes, creams and gels also may contain excipients, such as animal and vegetable fats, oils, waxes, paraffins, starch, tragacanth, cellulose derivatives, polyethylene glycols, silicones, bentonites, silicic acid, talc and zinc oxide, or mixtures thereof. Powders and sprays also can contain excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

The active compound may be mixed under sterile conditions with a pharmaceutically-acceptable carrier, and with any preservatives, buffers, or propellants which may be required. Typically, the topical formulations will comprise a preparation for delivering the active compound directly to the affected skin comprising the active compound together with a non-toxic, pharmaceutically acceptable topical carrier as set forth above. The active compound can be present in a suitable concentration that depends on the dosage form and amount, the particular active compound, the disorder being treated and the species or condition of the subject. See Dermatological Formulations: Percutaneous Absorption, Barry (ed.), Marcel Dekker Inc., (1983). For standard dosages of conventional pharmaceutical agents, see, e.g., Physicians Desk Reference 53rd Ed., Medical Economics Company, Montvale, N.J., USA 1999).

In the administration of the present compound by iontophoresis, electric gradients are used to enhance transdermal pharmaceutical agent delivery. Specifically, this technique uses an electric field to enhance the rate of delivery of ionized pharmaceutical agents through the skin or mucosal membranes. Typically, devices are used to hold the active compounds in a reservoir near the skin, generate an electric field surrounding the active compound-dermal interface, and drive the compound through the skin. These devices are known in the art. See Chien et al. Journal of Pharmaceutical Sciences 78, 353-354 (1989) and Banga et al., J. Controlled Release 7, 1-14 (1988). For general discussions of lontophoresis, see, e.g., Tyle, J., Pharm. Sd. 75, 318 (1989); Phipps et al., Solid State lonics 28, 1778-1783 (1988); and Phipps et al., J. Pharm. Sciences 78, 365-369 (1989).

Regardless of the particular method of administration, the amount of active compound administered will be dependent on the subject being treated, the severity and type of the disorder, the manner of administration and the judgment of the prescribing physician. For example, an effective daily dose for topical administration is about 1 mg/day to about 5 mg/day, and preferably about 2 mg/day, however, dosages as high as 20 mg/day may be administered. The daily dosage may be divided into multiple doses that are administered at various times throughout the day. For example, if a daily dosage of the active compound is 2 mg/day, the active compound may be administered in two doses per day of 1 mg each.

The invention is further defined by reference to the following examples. It will be apparent to those skilled in the art that many modifications, both to the materials and methods, may be practiced without departing from the purpose and interest of the invention.

### EXAMPLES

### Anti-proliferative Effects of 2′,3′-cyclophosphateadenylyl(2′,5′)adenylyl-(2′,5′)adenosine on Human Tumor Cells in Culture

2′,3′-cyclophosphateadenylyl(2′,5′)adenylyl-(2′,5′)adenosine has been found to have anti-proliferative activity against human papillomavirus-containing cell lines. In separate studies, 2′,3′-cyclophosphateadenylyl(2′,5′)adenylyl-(2′,5′)adenosine also inhibited the growth of Daudi tumor cells and normal human foreskin fibroblasts.

In the first study, the growth of human papillomavirus (HPV)-positive cell lines was measured in the presence of 2′,3′-cyclophosphateadenylyl(2′,5′)adenylyl-(2′,5′)adenosine or in the presence of a series of six known antiviral compounds including cidofovir (HPMPC; 3-hydroxy-2-phosphonylmethoxy-propyl cytosine). HPMPC is known to inhibit the proliferation of HPV-transformed cells. See e.g., G. Andrei et at., Oncol Res. 10, 523-31 (1998) and J. A. Johnson et al., Antimicrob. Agents Chemother. 43, 1198-205 (1999). With the exception of acyclovir, growth inhibition was observed for all compounds tested, but the data set forth herein relates to 2′,3′-cyclophosphateadenylyl(2′,5′)adenylyl-(2′,5′)adenosine and HPMPC.

The cells used in the first study included CK-1 (HPV-33-positive), EiL8 (HPV-16-positive), SiHa (HPV-16-positive) and HeLa (HPV-18-positive) and control primary human cervical keratinocytes (PHK). The cells were cultured in growth medium at 37° C., in 5% CO2. For these experiments, the cells were seeded in 96-well microtiter plates and after 24 hours of growth, serial dilutions of the test compounds were added. The cells were not fed after addition of the compounds. After three, five, seven and ten days, the cells were harvested by trypsinization and counted with a Coulter Counter. Duplicate wells were analyzed for each dilution and the results averaged. The number of cells recovered at each dilution was compared to the number of cells in untreated control wells and the concentration of test compound required to reduce cell growth by 50 percent (IC₅₀) was determined.

In a second study, 2′,3′-cyclophosphateadenylyl(2′,5′)adenylyl-(2′,5′)adenosine was found to inhibit the growth of Daudi tumor cells and normal human foreskin fibroblasts but not human embryonic lung fibroblasts. The human foreskin fibroblast (HFF) cells were cultured in growth medium as described above, but assayed by neutral red uptake and counting of cells. The human embryonic lung fibroblast (HEL) cells were cultured in growth medium and assayed as described above by counting with a Coulter Counter. Daudi cells, an Epstein-Barr Virus (EBV)-positive Burkitt's lymphoma tumor cell line, were cultured in growth medium as described above and assayed by the neutral red uptake assay. The anti-proliferative effects of 2′,3′-cyclophosphateadenylyl(2′,5′)adenylyl-(2′,5′)adenosine and HPMPC on HPV-containing cells are shown in Table 1, below. 2′,3′-cyclophosphateadenylyl(2′,5′)adenylyl-(2′,5′)adenosine also inhibited the growth of Daudi, an EBV-positive Burkitt's lymphoma tumor cell line, which illustrates the anti-proliferative effect of 2′,3′-cyclophosphateadenylyl(2′,5′)adenylyl-(2′,5′)adenosine on tumor cells of lymphoid origin. These results are shown in Table 2, below.

TABLE 1
Summary of the anti-proliferative effect of 2′,3′-cyclophosphateadenylyl
(2′,5′) adenylyl-(2′,5′) adenosine (Cmpd. I) on Primary Human
Keratinocytes and HPV⁺ Cell Lines

| Cell | Day 3 | Day 5 | | Day 7 | | Day 10 | | Number of |
|---|---|---|---|---|---|---|---|---|
| line | IC₅₀ (μg/ml)* | IC₅₀ (μg/ml)* | Ratio** | IC₅₀ (μg/ml)* | Ratio | IC₅₀ (μg/ml)* | Ratio | Repeats |
| PHK | 110 | 168 | 0.7 | 262 | 0.4 | 280 | 0.4 | 1 |
| Cmpd. I | | | | | | | | |
| HPMPC | 9.9 | 7.0 | 1.4 | 6.4 | 1.5 | 3.7 | 2.7 | 1 |
| HeLa | 66 | 62 | 1.1 | 90 | 0.7 | 160 | 0.4 | 2 |
| Cmpd. I | | | | | | | | |
| HPMPC | 37 | 5.6 | 6.6 | 3.9 | 9.5 | 4.7 | 7.9 | 2 |
| EiL8 | 63 | 68 | 0.8 | 103 | 0.5 | 180 | 0.3 | 2 |
| Cmpd. I | | | | | | | | |
| HPMPC | 39 | 7.9 | 4.9 | 4.2 | 9.3 | 3.5 | 11 | 2 |
| CK-1 | 100 | 46 | 2.2 | 104 | 1.0 | 303 | 0.3 | 1 |
| HPMPC | 32 | 1.1 | 29 | 0.5 | 64 | 1.2 | 27 | 1 |
| SiHa | 150 | 108 | 1.4 | 141 | 1.1 | 193 | 0.8 | 1 |
| Cmpd. I | | | | | | | | |
| HPMPC | 179 | 8.6 | 21 | 4.7 | 38 | 4.0 | 45 | 1 |

*Concentration required to reduce cell growth by 50%
**Ratio of IC₅₀ at day 3 to IC₅₀ at day 5, 7, or 10

TABLE 2
2′,3′-cyclophosphateadenylyl (2′,5′) adenylyl-(2′,5′) adenosine
inhibition of growth of human embryonic lung fibroblasts (HEL),
human foreskin fibroblasts (HFF), and Burkitt's
lymphoma cells (Daudi)

| | IC₅₀ |
|---|---|
| Cells | (μm/ml) |
| HEL | >1,000 |
| HFF | 46 |
| Daudi | >50 |

The foregoing is illustrative of the present invention and is not to be construed as limiting thereof. The invention is defined by the following claims, with equivalents of the claims to be included therein.

What is claimed is:

1. A method of treating a disorder of cell proliferation in a subject in need of such treatment, comprising administering to the subject a compound of Formula I or a pharmaceutically acceptable salt thereof: 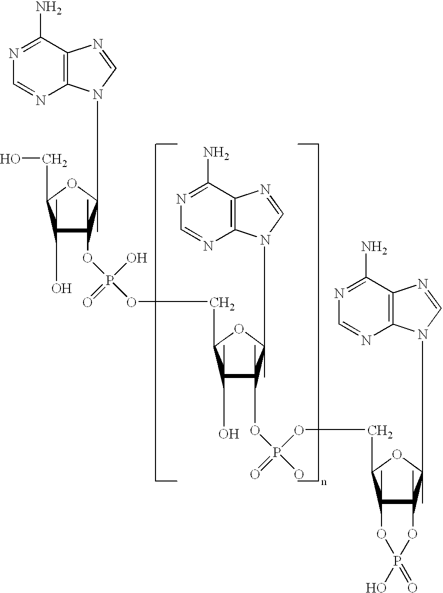
wherein 0≦n≦10, in an amount sufficient to treat the disorder of cell proliferation.

2. The method according to claim 1, wherein n is 1.

3. The method according to claim 1, wherein n is 2.

4. The method according to claim 1, wherein the subject is a human subject.

5. The method according to claim 1, wherein the disorder of cell proliferation is a hyperproliferative disorder.

6. The method according to claim 5, wherein the disorder of cell proliferation is cancer.

7. The method according to claim 6, wherein the cancer is basal cell carcinoma.

8. The method according to claim 6, wherein the cancer is squamous cell carcinoma

9. The method according to claim 5, wherein the hyperproliferative disorder is selected from the group consisting of hyperplasias, hyperpigmentation of the skin, psoriasis, Gardner syndrome, oral leukoplakias; histiocytoses, keloids, hyperproliferative arterial stenosis, hyperkeratoses, papulosquamous eruptions, and viral-induced hyperproliferative disorders.

10. The method according to claim 9, wherein the hyperproliferative disorder is a virus-induced hyperproliferative disorder.

11. The method according to claim 10, wherein the virus-induced hyperproliferative disorder is a hyperproliferative disorder induced by human papilloma virus.

12. The method according to claim 1, wherein the disorder of cell proliferation is selected from the group consisting genital warts, cutaneous warts, and lesions associated with human papilloma virus.

13. The method according to claim 1, wherein the disorder of cell proliferation is psoriasis.

14. The method according to claim 1, wherein the compound of Formula I or the pharmaceutically acceptable salt thereof is administered to the subject topically.

15. The method of claim 1, wherein the compound administered to the subject comprises an effective daily dosage in an amount from approximately 1 mg/day to 20 mg/day.

16. The method of claim 15, wherein the compound administered to the subject comprises an effective daily dosage of approximately 2 mg/day.

17. The method of claim 15, wherein the effective daily dosage may be administered in a single daily dosage or in multiple dosages throughout the day.

18. The method of claim 1, wherein the pharmaceutically acceptable salt is selected from the group consisting of alkali metal salts, alkaline earth metal salts, ammonium and tetra alkyl ammonium salt of the formula NX₄, wherein X is a C1-C4 alkyl group.

19. A method of treating cancer in a subject in need of such treatment, comprising administering to the subject a compound of Formula I or a pharmaceutically acceptable salt thereof: 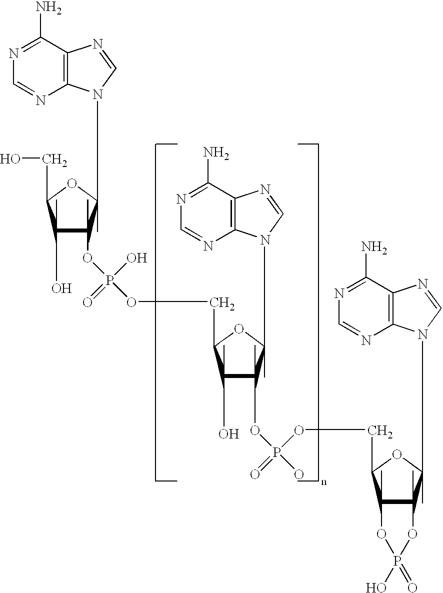
wherein 0≦n≦10, in an amount sufficient to treat the cancer.

20. The method according to claim 19, wherein n is 1.

21. The method according to claim 19, wherein n is 2.

22. The method according to claim 19, wherein the subject is a human subject.

23. The method according to claim 19, wherein the cancer is basal cell carcinoma.

24. The method according to claim 19, wherein the cancer is squamous cell carcinoma.

25. The method according to claim 19, wherein the subject has been diagnosed with cancer.

26. The method according to claim 19, wherein the subject is at risk of developing cancer.

27. The method according to claim 19, wherein the compound of Formula I or the pharmaceutically acceptable salt thereof is administered to the subject topically.

28. The method of claim 19, wherein the compound administered to the subject comprises an effective daily dosage in an amount from approximately 1 mg/day to 20 mg/day.

29. The method of claim 28, wherein the compound administered to the subject comprises an effective daily dosage of approximately 2 mg/day.

30. The method of claim 28, wherein the effective daily dosage may be administered in a single daily dosage or in multiple dosages throughout the day.

31. The method of claim 19, wherein the pharmaceutically acceptable salt is selected from the group consisting of alkali metal salts, alkaline earth metal salts, ammonium and tetra alkyl ammonium salt of the formula NX₄, wherein X is a C1-C4 alkyl group.

32. A method of decreasing tumor size in a subject in need of such treatment, comprising administering to the subject a compound of Formula I or a pharmaceutically acceptable salt thereof: 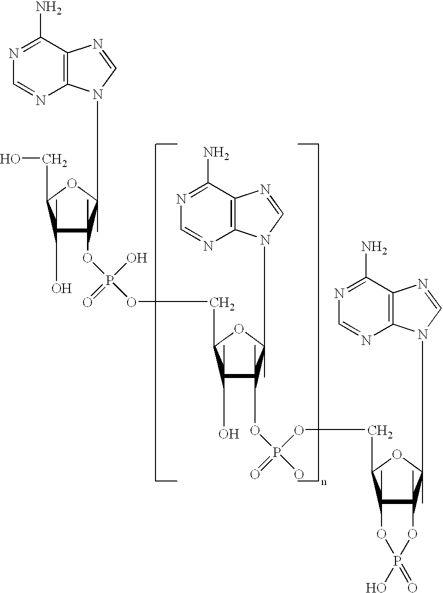
wherein 0≦n≦10, in an amount sufficient to decrease the tumor size.

33. The method according to claim 32, wherein n is 1.

34. The method according to claim 32, wherein n is 2.

35. The method according to claim 32, wherein the subject is a human subject.

36. The method according to claim 32, wherein the tumor is a skin tumor.

37. The method according to claim 36, wherein the tumor is a caused by a cancer selected from the group consisting of basal cell carcinoma and squamous cell carcinoma.

38. The method according to claim 32, wherein the tumor is malignant.

39. The method according to claim 32, wherein the tumor is benign.

40. The method according to claim 32, wherein the compound of Formula I or the pharmaceutically acceptable salt thereof is administered to the subject topically.

41. The method of claim 32, wherein the compound administered to the subject comprises an effective daily dosage in an amount from approximately 1 mg/day to 20 mg/day.

42. The method of claim 41, wherein the compound administered to the subject comprises an effective daily dosage of approximately 2 mg/day.

43. The method of claim 41, wherein the effective daily dosage may be administered in a single daily dosage or in multiple dosages throughout the day.

44. The method of claim 32, wherein the pharmaceutically acceptable salt is selected from the group consisting of alkali metal salts, alkaline earth metal salts, ammonium and tetra alkyl ammonium salt of the formula NX₄, wherein X is a C1-C4 alkyl group.
